(19) United States
(12) Patent Application Publication
(10) Pub. No.: US 2019/0060247 A1
(43) Pub. Date: Feb. 28, 2019

(54) MULTI DRUG COMPOSITE, METHODS AND USES THEREOF

(71) Applicant: Yissum Research Development Company, of The Herbrew University of Jerusalem Ltd., Jerusalem (IL)

(72) Inventors: Itamar WILLNER, Mevasseret-Zion (IL); Vered HELEG-SHABTAI, Hulda (IL); Ruth AIZEN, Tel Aviv (IL); Sharon ETERY, Rishon Le Tzion (IL); Natalie ENKIN, Mevasseret-Zion (IL)

(73) Assignee: Yissum Research Development Company, of The Hebrew University of Jerusalem Ltd., Jerusalem (IL)

(21) Appl. No.: 16/081,930

(22) PCT Filed: Mar. 2, 2017
(86) PCT No.: PCT/IL2017/050269

§ 371 Date: Sep. 3, 2018

Related U.S. Application Data
(60) Provisional application No. 62/302,895, filed on Mar. 3, 2016.

(51) Int. Cl. A61K 9/51; A61K 31/136; A61K 31/724; A61K 31/11; A61K 47/02; A61P 35/00

(52) U.S. Cl. CPC A61K 9/5192; A61K 9/5115; A61K 31/136; A61P 35/00; A61K 31/11; A61K 47/02; A61K 31/724

(57) ABSTRACT

The invention provides mesoporous nanoparticles loaded within its pores with at least one loaded pharmaceutically active agent and at least one capping pharmaceutically active agent chemically coordinated with at least one ligand being boronic acid or derivative thereof on the surface of said pores; compositions comprising such mesoporous nanoparticles and uses thereof.

### TECHNOLOGICAL FIELD

The present application discloses multiple drug composites, methods and uses thereof for the synergistic and cooperative treatment of diseases.

### BACKGROUND ART

References considered to be relevant as background to the presently disclosed subject matter are listed below:
1. (a) S. K. Nataraja and S. Stalin, RSC Adv., 2014, 4, 14328-14334; (b) M. C. Burleigh, S. Dai, E. W. Hagaman, C. E. Barnes and Z. L. Xue, ACS Symp. Series, 2001, 778, 146-158; (c) H.-T. Chen, S. Huh and V. S. Y. Lin, Catal. Prep., 2007, 45-73; (d) D. R. Radu, C.-Y. Lai, J. Huang, X. Shu and V. S. Y. Lin, Chem. Commun., 2005, 1264-1266; (e) D. R. Radu, C.-Y. Lai, J. W. Wiench, M. Pruski and V. S. Y. Lin, J. Am. Chem. Soc., 2004, 126, 1640-1641; (f) J. Lu, M. Liong, J. I. Zink and F. Tammanoi, Small, 2007, 3, 1341-1346; (g) X. Feng, G. E. Fryxell, L. Q. Wang, A. Y. Kim, J. Liu and K. M. Kemner, Science, 1997, 276, 923-926.
2. (a) F. Jiao and H. Frei, Angew. Chem., Int. Ed., 2009, 48, 1841-1844; (b) S. Huh, H.-T. Chen, J. W. Wiench, M. Pruski and V. S.-Y. Lin, Angew. Chem., Int. Ed., 2005, 44, 1826-1830; (c) S. Xiang, Y. Zhang, Q. Xin, C. Li, Chem. Commun., 2002, 2696-2697.
3. (a) K. S. Jang, H. J. Kim, J. R. Johnson, W. G. Kim, W. J. Koros, C. W. Jones and S. Nair, Chem. Mater., 2011, 23, 3025-3028; (b) R. Brady, B. Woonton, M. L. Gee and A. J. O'Connor, Innovative Food Sci. Emerging Technol., 2008, 9, 243-248.
4. (a) M. Vallet-Regi, F. Balas and D. Arcos, Angew. Chem., Int. Ed., 2007, 46, 7548-7558; (b) M. Liong, J. Lu, M. Kovochich, T. Xia, S. G. Ruehm, A. E. Nel, F. Tamanoi and J. I. Zink, ACS Nano, 2008, 2, 889-896.
5. (a) J. Kim, H. S. Kim, N. Lee, T. Kim, H. Kim, T. Yu, I. C. Song, W. K. Moon and T. Hyeon, Angew. Chem., Int. Ed., 2008, 47, 8438-8441; (b) C.-P. Tsai, Y. Hung, Y.-H. Chou, D.-M. Huang, J.-K. Hsiao, C. Chang, Y.-C. Chen and C.-Y. Mou, Small, 2008, 4, 186-191;
6. (a) S-H. Wu, C-Y. Mou and H-P. Lin, Chem. Soc. Rev., 2013, 42, 3862; (b) Q. Cai, Z. S. Luo, W. Q. Pang, Y. W. Fan, X. H. Chen and F. Z. Cui, Chem. Mater., 2001, 13, 258-263; (c) C. E. Fowler, D. Khushalani, B. Lebeau and S. Mann, Adv. Mater., 2001, 13, 649-652; (d) R. I. Nooney, D. Thirunavukkarasu, Y. M. Chen, R. Josephs and A. E. Ostafin, Chem. Mater., 2002, 14, 4721-4728.
7. C.-Y. Lai, B. G. Trewyn, D. M. Jeftinija, K. Jeftinija, S. Xu, S. Jeftinija and V. S.-Y. Lin, J. Am. Chem. Soc., 2003, 125, 4451-4459.
8. R. Liu, X. Zhao, T. Wu and P. Feng, J. Am. Chem. Soc., 2008, 130, 14418-14419.
9. (a) A. Yu, Y. Wang, E. Barlow and F. Caruso, Adv. Mater., 2005, 17, 1737-1741; (b) Y. Zhu, J. Shi, W. Shen, X. Dong, J. Feng, M. Ruan and Y. Li, Angew. Chem., Int. Ed., 2005, 44, 5083-5087; (c) Q. Yang, S. Wang, P. Fan, L. Wang, Y. Di, K. Lin and F.-S. Xiao, Chem. Mater., 2005, 17, 5999-6003; (d) Q. Gao, Y. Xu, D. Wu, W. Shen and F. Deng, Langmuir, 2010, 26, 17133-17138; (e) H. Zheng, Y. Wang and S. Che, J. Phys. Chem. C, 2011, 115, 16803-16813.
10. (a) C. Park, K. Oh, S. C. Lee and C. Kim, Angew. Chem., Int. Ed., 2007, 46, 1455-1457; (b) C. Park, H. Kim, S. Kim and C. Kim, J. Am. Chem. Soc., 2009, 131, 16614-16615.
11. (a) L. Du, S. Liao, H. A. Khatib, J. F. Stoddart and J. I. Zink, J. Am. Chem. Soc., 2009, 131, 15136-15142. (b) S. Angelos, M. Liong, E. Choi and J. I. Zink, Chem. Eng. J, 2008, 137, 4-13.
12. E. Climent, R. Martinez-Manez, F. Sancenon, M. D. Marcos, J. Soto, A. Maquieira and P. Amoros, Angew. Chem., Int. Ed., 2010, 49, 7281-7283.
13. (a) D. Liu and S. Balasubramanian, Angew. Chem. Int. Ed., 2003, 42, 5734-5736; (b) S. Modi, M. G. Swetha, D. Goswami, G. D. Gupta, S. M. Krishnan, Nat. Nanotechnol., 2009, 4, 325-330; (c) A. Idili, A. Vallee-Belisle and F. Ricci, J. Am. Chem. Soc., 2014, 136, 5836-9583; (d) T. Li and M. Famulok, J. Am. Chem. Soc., 2013, 135, 1593-1599.
14. Z. Zhang, F. Wang, Y. S. Sohn, R. Nechushtai and I. Willner, Adv. Funct. Mater., 2014, 24, 5662-5670.
15. Z. Zhang, D. Balogh, F. Wang, S. Y. Sung, R. Nechushtai and I. Willner, ACS Nano, 2013, 7, 8455-8468.
16. (a) B. Hildebrandt, P. Wust, O. Ahlers, A. Dieing, G. Sreenivasa, T. Kerner, R. Felix and H. Riess, Crit. Rev. Oncol. Hematol., 2002, 43, 33-56; (b) J. Croissant and J. I. Zink, J. Am. Chem. Soc., 2012, 134, 7628-7631.
17. Z. Zhang, D. Balogh, F. Wang and I. Willner, J. Am. Chem. Soc., 2013, 135, 1934-1940.
18. (a) A. Aravind, S. Veeranarayanan, A. C. Poulose, R. Nair, Y. Nagaoka, Y. Yoshida, T. Maekawa and D. S. Kumar, BioNanoSci., 2012, 2, 1-8; (b) H. Li, Y. Mu, S. Qian, J. Lu, Y. Wan, G. Fua and S. Liu, Analyst, 2015, 140, 567.
19. R. V. Smalley, S. Murphy, C. M. Huguley, Jr. and A. A. Bartolucci. Cancer Res, 1976, 36, 3911-3916.
20. S. Kitada, M. Leone, S. Sareth, D. Y. Zhai, J. C. Reed and M. Pellecchia, J. Med. Chem., 2003, 46, 4259-4264; (b) R. M. Mohammad, S. Wang, A. Aboukameel, B. Chen, X. Wu, J. and A. Al-Katib, Chen, Mol. Cancer Ther., 2005, 4, 13-21; (c) C. L. Oliver, M B. Miranda, S Shangary, S. Land, S. Wang and D E. Johnson, Mol. Cancer Ther., 2005, 4, 23-31; (d) S. W. Fesik, Nature Reviews Cancer, 2005, 5, 876-885; (e) F. Hu, K. Mah and D. J., Teramura, In Vitro Cell Dev Biol. 1986, 22, 583-588; (f) G. P., Tuszynski and G. Cossu, Cancer Res., 1984, 44, 768-771; (g) W. S. Yeow, A. Baras, A. Chua, D. M. Nguyen, S. S. Sehgal, D. S. Schrump and D. M. Nguyen, J Thorac Cardiovasc Surg., 2006, 132, 1356-1362; (h) K. Dodou, R. J. Anderson, D. A. Small and P. W. Groundwater, Expert Opin. Invest. Drugs, 2005, 14, 1419-1434; (i) Y. Yuan, A. J. Tang, A. B. Castoreno, S. Y. Kuo, Q. Wang, P. Kuballa, R. Xavier, A. F. Shamji, S. L. Schreiber and B. K. Wagner, Cell Death Dis., 2013, 4, e690.
21. M. Mego, Bratisl. Lek. Listy., 2002, 103, 378-381.
22. G. S. Kwon, H-C. Shin and H. Cho, “Micelles for the solubilization of gossypol” Patent US20120321715 A1. 20 Dec. 2012.
23. V. Heleg-Shabtai, R. Aizen, R. Orbach, M. A. Aleman-Garcia and I. Willner. Langmuir, 2015, 31, 2237-2242.
24. P. M. Carli, C. Sgro, N. Parchin-Geneste, N. Isambert, F. Mugneret, F. Girodon and M. Maynadie, Leukemia, 2000, 14, 1014-1017.
25. G. Springsteen and B. Wang, Tetrahedron, 2002, 58, 5291-5300.
26. W. Yang, X. Gao and B. Wang, Boronic Acids: Preparation and Applications in Organic Synthesis and Medicine; Hall, D. G., Ed.; WileyVCH: Weinheim, Germany, 2005.
27. S. Zhou, H. Sha, X. Ke, B. Liu, X. Wang and X. Du. Chem. Commun., 2015, 51, 7203-7206.
28. Y. Zhao, B. G. Trewyn, I. I. Slowing and V. S.-Y. Lin, J. Am. Chem. Soc., 2009, 131, 8398-8400.
29. M. M. Leane, R. Nankervis, A. Smith and L. Ilium, Int. J. Pharm., 2004, 271, 241-249.
30. G. Springsteen and B. Wang. Chem. Commun., 2001, 1608-1609.
31. J. R. Griffiths, Br. J. Cancer, 1991, 64, 425-427.
32. F. K. Sartain, X. Yang and C. R. Lowe, Chem.-Eur. J., 2008, 14, 4060-4067.
33. (a) H. Meng, M. Liong, T. Xia, Z. Li, Z. Ji, J. I. Zink and A. E. Nel, ACS Nano, 2010, 4, 4539-4550; (b) Z. Li, D. L. Clemens, B-Y. Lee, B. J. Dillon, M. A. Horwitz and J. I. Zink. ACS Nano, 2015, 9, 10778-10789.
34. (a) A. M. Chen, M. Zhang, D. G. Wei, D. Stueber, O. Taratula, T. Minko and He. Huixin, Small, 2009, 5, 2673-2677; (b) D. P. Ferris, J. Lu, C. Gothard, R. Yanes, C. R. Thomas, J. C. Olsen, J. F. Stoddart, F. Tamanoi and J. I. Zink, Small, 2011, 7, 1816-1826.
35. G. R. Nakayama, M. C. Caton, M. P. Nova and Z. Parandoosh. Journal of Immunological Methods, 1997, 204, 205-208.

Acknowledgement of the above references herein is not to be inferred as meaning that these are in any way relevant to the patentability of the presently disclosed subject matter.

### BACKGROUND

Mesoporous silica attracts growing interest due to its high surface area and the ability to modify its surface.¹ Different applications of this mesoporous silica were suggested, including the development of catalysts,² separation,³ delivery⁴ and imaging materials.⁵ Different methods for synthesising mesoporous silica nanoparticles, MP-SiO₂ NPs, and their functionalization to yield stimuli-responsive NPs were reported.⁶ In these systems, the pores of the MP-SiO₂ NPs are loaded with substrates and capped by stimuli-responsive caps. In the presence of appropriate triggers, the caps are unlocked, thus allowing the controlled release of the loaded substrates. Different triggers such as photonic signals,⁷ redox signals,⁸ pH⁹ or enzymes¹⁰ were used to unlock the pores, and release the entrapped loads. Also, supramolecular structures acting as molecular machines (valves) were used to lock the pores and to stimulate the unlocking of the pores by chemical stimuli.¹¹ Alternatively, substrate-loaded MP-SiO₂ NPs were capped with nucleic acid nanostructures and the DNA caps were unlocked by their signal-triggered reconfiguration,¹² e.g., by pH,¹³ K⁺/ligands,¹⁴ formation of aptamer-ligand complexes,¹⁵ or by catalytic degradation, e.g., by enzymes^{15,16} or DNAzymes.¹⁷ These stimuli-responsive MP-SiO₂ NPs find major applications for controlled drug delivery, such as anti-cancer drugs (doxorubicin or camptothecin). In these systems, the over-expression of ATP in cancer cells, the slightly acidic pH of cancer cells, and cancer-cell specific enzymes, e.g β-galactosidase were used as environmental triggers for the selective “unlocking” of the drug-loaded MP-SiO₂ NPs. Also, the surface modification of the MP-SiO₂ NPs with cancer cell-specific aptamers, e.g., AS1411 allowed the targeting of cancer cells and facile intracellular release of the drug loaded.¹⁸

### GENERAL DESCRIPTION

The present invention provides mesoporous nanoparticle (MP-NP) loaded within its pores with at least one loaded pharmaceutically active agent; comprising at least one ligand, being boronic acid or a derivative thereof, on the surface of said pores chemically coordinated with at least one capping pharmaceutically active agent.

The term “mesoporous nanoparticle (MP-NP)” should be understood to encompass any type of nanoparticle from any type of material having mesoporous three dimensional structure containing pores with diameters between 2 and 50 nm.

In some embodiments, said MP-NP is being selected from silica, alumina, zirconia, titania, carbon nanoparticle, and any combinations thereof.

Said MP-NP of the invention comprise within its pores at least one loaded pharmaceutically active agent, thus entrapping said at least one loaded active agent within the pores of said MP-NP. Each pore of said MP-NP can accommodate at least one loaded pharmaceutically active agent.

When referring to at least one loaded pharmaceutically active agent and/or to said at least one capping pharmaceutically active agent it should be understood to encompass an active agent that is acting as a pharmaceutical drug for the treatment of a disease, disorder or a symptom of a subject. Since the MP-NP of the present invention can carry multiple pharmaceutically active agents, it is possible to design an MP-NP carrying complementary active agents that can benefit the treatment of a disease, disorder or symptom thereof in a synergistic manner. The activity of the carried active agents can be selected so that the therapeutic benefits of the treatment will be enhanced due to the simultaneous release of the agents at the target site of treatment.

In some embodiments said at least one loaded pharmaceutically active agent and and said at least one capping pharmaceutically active agent are selected for use in the treatment of cancer.

In further embodiments, said at least one loaded pharmaceutically active agent is mitoxantrone or any combinations thereof.

As defined, said MP-NP of the invention comprise at least one ligand on the surface of said pores, being a boronic acid or derivative thereof. Thus, the surface of said MP-NP, for example on the surface of the outer rim, edge, boarder of the circumference of the pore, is chemically modified with these functionalized ligands, specifically on the outer surface of the pores of said MP-NP. The uniqueness of boronic acid and derivatives thereof provides a dual purpose. On the one hand boronic acid is capable of forming chemical coordinative bonds with said at least one capping pharmaceutically active agent thereby forming a cap over the pores of said MP-NP loaded with said at least one loaded pharmaceutically active agent, maintaining the loaded agents inside the pores of the MP-NP and enabling the MP-NP to carry both said at least one capping pharmaceutically active agent and said at least one loaded pharmaceutically active agent. On the second hand, said boronic acid and derivatives thereof react competitively with markers found is specific target cells for which the treatment is designed for, thereby releasing the carried active agents on said MP-NP.

In some embodiments, the boronic acid or derivative thereof is capable of releasing said capping pharmaceutically active agent upon reaching a target cell having a specific pH. Some cancer cells have a particular acidic local environment which triggers the release of said at least one capping pharmaceutically active agent and at the same time opening the pores of said MP-NP releasing said at least one loaded pharmaceutically active agent. In other cases, some cancer target cells are biochemically over expressing the production of lactic acid. The over expression of lactic acid is able to trigger the release of said at least one capping pharmaceutically active agent from the coordinative bond with the boronic acid and at the same time opening the pores of said MP-NP releasing said at least one loaded pharmaceutically active agent

In some embodiments, said MP-NP comprises at least one further ligand of the surface of said NP (i.e. not only on the surface of said pore). Said at least one further ligand (being the same or different from said at least one ligand) are capable of chemically anchoring at least one further agent being selected from a targeting agent, a solubilizing agent, a protein, a carbohydrate and so forth. Such further agents are also released from the coordinative chemical bond with the ligand at the target cell due to specific target markers. Such further agents can include for example saccharides, aptamers, proteins and are able to enhance the solubility of the released pharmaceutically active agents, enhance the targeting of said MP-NP and also may add therapeutic activity (such as for example further cytotoxicity).

The MP-NP of the invention further comprises at least one capping pharmaceutically active agent which is chemically coordinated with said at least one ligand on the surface of said pore. Said chemical coordination of ligand and capping active agent includes any type of chemical coordination bond, including but not limited to a hydrogen bond, an electron bond, a salt bond, a π-π bond, a σ-π bond, a metal coordination bond, a σ bond, a π bond or any combination thereof.

Said at least one capping pharmaceutically active agent upon its attachment to said ligand on the surface of said pore of MP-NP is able to cap the pore, thus entrapping said at least one loaded active agent within the pore of said MP-NP. The triggered release of both agents is executed upon competitive coordination of said ligand or at least one capping agent, with a biochemical agent at a target location.

In some embodiments, said MP-NP of the invention is capable of triggered simultaneous release of said at least one loaded pharmaceutically active agent and said at least one capping pharmaceutically active agent.

In other embodiments, said MP-NP of any one of the preceding claims, capable of triggered simultaneous controlled release of said at least one loaded pharmaceutically active agent and said at least one capping pharmaceutically active agent.

In other embodiments, said at least one capping pharmaceutically active agent is selected from gossypol, cyclodextrin and any derivatives thereof or any combinations thereof.

The invention further provides a composition comprising at least one MP-NP as disclosed herein above and below.

The invention further provides a composition as disclosed herein above and below for use in the treatment of at least one disease, disorder or symptom thereof.

In some embodiments, said at least one disease, disorder or symptom thereof is cancer.

It is important to note that cancer treatment benefits from combined drug approach taking advantage of synergistic effect of at least two active agents (one or both being anti-caner agents). Research has shown that combined therapies, especially those administered at the target cell are highly effective and provide extremely favorable results with respect to treatment of the disease and limitation of the well known side effects of cytotoxic drugs. Understanding this approach makes the MP-NP of the present invention a tailored carrier of at least two active agents (at least one loaded pharmaceutically active agent and at least one pharmaceutically active capping agent) suitable for effective treatment of cancer. Thus, in some embodiments a MP-NP of the present invention comprises an anti-cancer agent as the at least one loaded pharmaceutically active agent. In some further embodiments a MP-NP of the present invention comprises at least one capping pharmaceutically active agent is an anti-cancer agent capable of forming a coordinative bond with the boronic acid ligand. In other embodiments, a MP-NP of the present invention comprises gossypol or any derivative thereof as said at least one capping pharmaceutically active agent. In further embodiments, MP-NP of the present invention comprises cyclodextrin or any derivative thereof as said at least one capping pharmaceutically active agent. In some embodiments said cyclodextrin further carries another it is complexed with. In some further embodiments, said MP-NP comprises cyclodextrin or any derivative thereof as said at least one capping pharmaceutically active agent and at least one further ligand of the surface of said NP (i.e. not only on the surface of said pore). Said at least one further ligand (being the same or different from said at least one ligand) are capable of chemically anchoring at least one further agent being selected from a targeting agent, a solubilizing agent, a protein, a carbohydrate and so forth. Such further agents are also released from the coordinative chemical bond with the ligand at the target cell due to specific target markers. Such further agents can include for example saccharides, aptamers, proteins and are able to enhance the solubility of the released pharmaceutically active agents, enhance the targeting of said MP-NP and also may add therapeutic activity (such as for example further cytotoxicity).

In a further aspect the invention provides a MP-NP comprising at least one ligand, being boronic acid or a derivative thereof, on the surface of said MP-NP chemically coordinated with at least one gossypol molecule. In some embodiments said MP-NP further comprising at least one pharmaceutically active agent loaded within the pores of said MP-NP. In other embodiments, said MP-NP further comprising at least one ligand on the surface of said MP-NP chemically coordinated with at least one further active agent. In some embodiments, said further active agent is selected from a targeting agent, a solubilizing agent, an aptamer, a protein, a carbohydrate and any combinations thereof.

In another aspect the invention provides a MP-NP comprising at least one ligand bring boronic acid or derivative thereof on the surface of said pores chemically coordinated with at least one cyclodextrin molecule. In some embodiments said MP-NP, further comprising at least one pharmaceutically active agent loaded within the pores of said MP-NP. In further embodiments, said MP-NP further comprises at least one ligand on the surface of said MP-NP chemically coordinated with at least one further active agent. In some embodiments, said further active agent is selected from a targeting agent, a solubilizing agent, an aptamer, a protein, a carbohydrate and any combinations thereof.

Mesoporous SiO₂ nanoparticles, MP-SiO₂ NPs, can be functionalized with the boronic acid ligand units. The pores of the MP-SiO₂ NPs are loaded with the anti-cancer drug mitoxantrone, and the pores are capped with the anti-cancer drug gossypol. The resulting two-drug-functionalized MP-SiO₂ NPs provide a potential stimuli-responsive anti-cancer drug carrier for cooperative chemotherapeutic treatment. In vitro experiments reveal that the MP-SiO₂ NPs are unlocked under environmental conditions present in cancer cells, e.g., pH acidic pH and added lactic acid over-expressed in cancer cells. The effective unlocking of the capping units under these conditions is attributed to the acidic hydrolysis of the boronate ester capping units and to the cooperative separation of the boronate ether bridges by the lactate ligand. The drug-loaded MP-SiO₂ NPs reveal impressive long-term stabilities.

The preset invention discloses the use of phenylboronic acid-modified MP-SiO₂ NPs as functional nano-container matrices, for the trapping of two anti-cancer drugs: gossypol (1) and mitoxantrone, MX (2), FIG. 1. The inventors disclose the pH/lactic acid cooperative “unlocking” of the MP-SiO₂ NPs and the release of the two drugs. The inventors further examined the cytotoxicity of the gossypol-capped mitoxantrone-loaded MP-SiO₂ NPs and their effect on MCF-10A breast cells and MDA-MB-231 breast cancer cells, respectively.

The cooperative activity of mixtures of anti-cancer drugs attracts interest as an improved method for combination chemotherapeutic treatments. This approach provides a higher probability to destroy cancer cells, as in the case of the treatment of metastatic carcinoma of the breast cancer.¹⁹ Developing stimuli-responsive drug carriers that deliver two or more anti-cancer drugs specifically into cancer cells, with limited toxicity toward normal cells, is still a challenge. Gossypol (1) is a natural phytochemical pigment extracted from cotton plants that attracts recent interest as a potential anti-cancer drug.²⁰ Specifically, it has been demonstrated that gossypol induces apoptosis of prostate cancer cells and reveals potential telomerase inhibition functions.²¹ Its chemotherapeutic use is, however, hampered due to low water solubility and cytotoxic side effects. Methods to facilitate the solubilization of gossypol in water by means of micelles or synthetic polymers were reported.²² Also, the caging of gossypol in hydrogel matrices and the dissolution of the hydrogel under acidic conditions were reported.²³ Mitoxantrone (2) is an anthraquinone derivative that is used as chemotherapeutic drug for the treatment of certain types of cancer, such as breast cancer, acute leukemia and lymphoma.²⁴ Thus, the invention provides, in one of its embodiments, a gossypol-capped mitoxantrone-loaded MP-SiO₂ NPs as a stimuli-responsive material for the controlled concomitant release of the two chemotherapeutic drugs.

Boronic acid ligands bind to vicinal cis-diols through the formation of boronate ester complexes.²⁵ Boronic acid esters are hydrolyzed under acidic conditions²⁶ or undergo, in the presence of other cis-vicinal diols, ligand exchange. Indeed, substrate-loaded MP-SiO₂ NPs capped with γ-cyclodextrin were “unlocked” under acidic conditions.²⁷ Similarly, Adenosine monophosphates (AMP)-loaded MP-SiO₂ NPs were capped with glucose-modified insulin and the pores were unlocked in the presence of monosaccharides (by ligand exchange) or acidic pH, to release the AMP-load.²⁸ The fact that gossypol (1) is a macrocycle, consisting of bidentante-o-dihydroxybenzene moieties suggests that it could function as a cap bridging boronic acid ligands associated with the MP-SiO₂ NPs. Accordingly, FIG. 1 outlines the preparation of the gossypol-capped mitoxantrone-loaded MP-SiO₂ NPs and the principle of unlocking the modified NPs and release of the two chemotherapeutic drugs: mitoxantrone and gossypol.

MP-SiO₂ NPs, as well as the functionalization steps were prepared according to the reported procedure.²⁸ The NPs were modified by aminopropyl siloxane units by the reaction with 3-aminopropyltrimethoxysilane. The resulting amine-functionalized mesoporous silica nanoparticles, were modified with p-carboxyphenylboronic acid to yield the phenylboronic acid (BA) ligand functionalized NPs, BA-MP-SiO₂ NPs. The diameter of the resulting NPs corresponded to ca. 250-350 nm. The coverage of the amine-functionalities on the MP-SiO₂ NPs was evaluated by the ninhydrin test²⁹ to be 5.7 nmole·gr⁻¹. The subsequent modification of the surface of the NPs by the boronic acid ligands was characterized by two methods: i) A qualitative method based on the reaction of the boronic acid ligands with Alizarin Red S. ii) A quantitative evaluation based on ninhydrin test.

Alizarin Red S binds to boronic acid ligand and the resulting boronate ester reveals a spectral shift.³⁰ FIG. 2 depicts the absorption spectrum of Alizarin Red S in solution λₘₐₓ=520 nm, curve (a). Treatment of the amine-modified MP-SiO₂ NPs with Alizarin Red S leads to a minute spectral shift, curve (b). In turn, treatment of the BA-MP-SiO₂ NPs with Alizarin Red S results in a pronounced blue-shift in the absorption spectrum, λₘₐₓ=480 nm, curve (c), implying that the dye, indeed, binds to the boronic acid ligands. The quantitative evaluation of the coverage of the boronic acid ligands associated with the BA-MP-SiO₂ NPs was evaluated by subjecting the amine-modified MP-SiO₂ NPs and the BA-MP-SiO₂ NPs to the ninhydrin test³³. By subtracting the amount of remaining surface amine groups on the BA-MP-SiO₂ NPs from that on amine-modified mesoporous SiO₂ surface, it was estimated that the surface coverage of the boronic acid ligands corresponds to ca. 1.6 nmole·gr⁻¹, indicating that ca. 28% of the amine functionalities associated with the NPs were modified by the boronic acid ligands.

BET measurements were further implemented to characterize the surface features (surface area, pore volume and pore diameter) of the NPs, upon their stepwise surface modification. The surface features of the modified particles are summarized in Table 1. The chemical modification of the “bare” MP-SiO₂ NPs with the aminopropyl functionalities, and subsequently, with the boronic acid ligands, consistently decrease the surface area of the NPs and reduces the pore volume and pore diameter of the NPs. These results are consistent with the functionalization of nanoporous domains and the inner-pore walls by the chemically modified ligands.

TABLE 1
The surface features of the mesoporous SiO₂ NPs, the
aminopropyl-modified MP SiO₂ NPs and the boronic
acid-functionalized MP SiO₂ NPs.

| | BET | BET | BJH |
|---|---|---|---|
| | Surface area | Pore Volume | Pore diameter |
| Material | m²/g | cm³/g | WBJH (nm) |
| MP-SiO₂ | 948.2021 | 0.881511 | 3.126 |
| NH₂-MP-SiO₂ | 630.8983 | 0.494471 | 2.5661 |
| BA-MP-SiO₂ | 617.1121 | 0.41905 | 2.4324 |

The loading of the BA-MP-SiO₂ NPs and the stimuli-controlled release of the pore-entrapped substrates are depicted in FIG. 1. The BA-MP-SiO₂ NPs were loaded with the anti-cancer drug mitoxantrone, MX (2) or with the model substrate methyene blue, MB⁺ (3). The loaded NPs were capped with gossypol (1), and the MB⁺ or MX substrates associated with surface domain or uncapped pores were intensively washed off. The loadings of MB⁺ or MX in the gossypol-capped MP-SiO₂ NPs were evaluated by measuring the absorption spectra of the suspended loaded NPs. Using this method, the loadings of MB⁺ or MX in the gossypol-capped MP-SiO₂ NPs were estimated to be 12.2 μmol·gr⁻¹, and 31.5 μmol·gr⁻¹, respectively. The unlocking of the gossypol-capped MP-SiO₂ NPs and the subsequent release of MB⁺ or MX was, then, examined under conditions that could stimulate the unlocking process in cancer cell environments: (i) Cancer cells reveal an acidic environment as compared to normal cells.³¹ The boronate ester groups are subjected to hydrolysis under acidic conditions, and thus, the dissociation of the gossypol-bridged boronate esters could provide a mechanism to unlock the pores and release the loads from the gossypol-capped MP-SiO₂ NPs; (ii) Lactic acid is over-expressed in cancer cells, due to the high rate of glycolysis followed by lactic acid formation. Enhanced hydrolysis of boronate esters by lactic acid as compared to formic acid at identical pH environments was demonstrated.³² This was attributed to the ligand exchange of the boronic acid residues by α-hydroxy carboxylic acids (such as lactic acid), a process that acts cooperatively with the pH-stimulated cleavage of the boronate ester bonds. That is, the acidic pH in cancer cells and the over-expressed lactic acid generated in malignant cells could act cooperatively in the unlocking of the gossypol-capped drug-loaded MP-SiO₂ NPs. These cooperative effects could induce selective unlocking of the gossypol-capped drug-loaded MP-SiO₂ NPs in cancer cells over normal cells. Accordingly, the effect of pH on the efficiency of unlocking of the gossypol-capped drug-loaded MP-SiO₂ NPs was examined and the cooperative effect of the pH and lactic acid on the dissociation of the gossypol-capped NPs and the efficiency of drug release.

FIG. 3(A) depicts the time-dependent fluorescence changes of MB⁺ upon unlocking the MB⁺-loaded gossypol-capped MP-SiO₂ NPs at different pH values and upon the implementation of the cooperative unlocking of the NPs by pH and lactic acid. In a phosphate-buffered saline solution, PBS, 200 mM, at pH=7.4, very inefficient release of MB⁺ is observed. Treatment of the MB⁺-loaded gossypol-caped NPs with lactic acid, 200 mM, at pH=6.0 results in the effective release of MB⁺, curve (b). It should be noted that in the presence of formate at pH=6 the release of MB⁺ was inefficient. Treatment of the MB⁺-loaded gossypol-caped NPs with formic acid, 200 mM, at pH=4.5, results in the release of MB⁺ from the pores at an efficiency and rate that are very similar to the release of MB⁺ from the NPs using lactic acid, 200 mM, at pH=6.0 as unlocking agent, curve (c). Upon the application of lactic acid as unlocking agent at pH=4.5, faster and more efficient release of MB⁺ are observed, curve (d). Similar results are observed for the release of MX from the MX-loaded gossypol-capped NPs, FIG. 3(B). The lactic acid-stimulated release of MX at pH=6.0 is efficient and reveals similar efficiency to that obtained with formic acid at pH=4.5. Similarly, the release of MX in the presence of lactic acid at pH=4.5 is more efficient than the release stimulated by formic acid at the same pH. These results clearly indicate that the activity of the unlocking agent, lactic acid, which provides an effective means to unlock the NPs and release the entrapped constituents, is following two mechanisms: ligand exchange and hydrolysis in acidic pH. The loading of the gossypol-capped mitoxantrone-loaded MP-SiO₂ NPs samples could be reproduced with an accuracy of ±5% in N=4 experiments. The gossypol-capped mitoxantrone-loaded MP-SiO₂ NPs revealed impressive stability in the dry state or in a PBS solution, upon storage at 4° C. It was find that the properties of the drug-loaded NPs are unchanged during a time-interval of two months.

In order to mimic the environmental conditions present in the cancer cell environment, the lactic acid-triggered drug release at pH=6.0, was characterized. It was estimated that the degree of release of MB⁺ or MX from their respective absorbance spectra using a defined composition of gossypol capped MP-SiO₂ NPs in the presence of lactic acid, 200 mM, pH=6.0, at 37° C. and after a time-interval of 24 hours. The MP-SiO₂ NPs loaded with 12.2 μmole·gr⁻¹ of MB⁺ were found to release 9.5 μmole·gr⁻¹ of MB⁺ into the solution, a value that corresponds to ca. 78% of the loaded content. Similarly, the gossypol-capped MX-loaded NPs loaded with 31.5 μmole·gr⁻¹ of MX released 19.8 μmole·gr⁻¹ of MX. This corresponds to the release of ca. 63% of the MX entrapped in the NPs. The incomplete release of the loads is attributed to the entrapment of the loads in nanopore domains that prohibit the escape of the loads to the bulk solution, or result in very slow release of the residual loads. This incomplete or very slow release phenomenon was observed with other molecular loads bound to mesoporous SiO₂ NPs.³³

The characterization of the gossypol-capped MX-loaded NPs, and the lactic-acid stimulated unlocking of NPs and release of gossypol/MX, encouraged us to probe the in vitro effects of the drug loaded NPs on cancer cells. In order to elucidate the functions of the combination of two anti-cancer drug on the cell viability a control system had to be designed so that it involves the release of MX alone, using an analogous release mechanism. β-Cyclodextrin is a macromolecular oligosaccharide structure consisting of a circle of seven glucose units linked via a 1-4 β-glycoside bond. The glucose units include vicinal diol functionalities capable of forming boronate ester bonds with the phenylboronic acid ligands associated with the modified MP-SiO₂ NPs. Accordingly, the boronic acid-functionalized MP SiO₂ NPs were loaded with MX, and capped the pores with β-cyclodextrin, β-CD, FIG. 4(A). The loading of the MX in the MP-SiO₂ NPs was estimated to be 22.1. FIG. 4 (B) depicts the release of the MX from the β-CD-capped MX-loaded MP-SiO₂ NPs at different pH values and upon the implementation of the cooperative unlocking of the NPs by pH and lactic acid. At pH=7.4 (in a PBS solution, 200 mM) the pores are not unlocked, curve (a). In the presence of lactic acid, 200 mM, at pH=6.0, the pores are unlocked and MX is released from the pores, curve (b). Treatment of the MX-loaded MP-SiO₂ NPs with formic acid, 200 mM, at pH=4.5, results in the release of MX from the pores at an efficiency and rate that are very similar to the release of MX from the NPs using lactic acid, 200 mM, at pH=6.0 as unlocking agent, curve (c). In the presence of lactic acid, 200 mM, at pH=4.5, the release of the drug is more faster and more efficient than the release stimulated by formic acid at the same pH, curve (d).

From the absorbance intensity of the released MX, it was estimated that after 24 h, 16.9 μmole·gr⁻¹ of MX are released from the NPs. Thus, the MX-loaded MP-SiO₂ NPs are being unlocked under conditions available in cancer cells, and, hence, provides a useful, model system, for the release of the single chemotherapeutic drug, MX. This corresponds to the release of ca. 76% of the MX entrapped in the NPs.

The present invention has introduced a method to assemble a stimuli-responsive drug carrier composed of mesoporous SiO₂ NPs loaded with two anti-cancer drugs for cooperative chemotherapeutic treatment. The MP-SiO₂ NPs carriers consist of gossypol-capped mitoxantrone-loaded NPs. The release of the drugs from the NPs is stimulated by unlocking of the gossypol caps under environmental conditions present in cancer cells. These include an acidic environment and the presence of over-expressed lactic acid. The acidic conditions allow the hydrolytic cleavage of the boronate ester groups linking the gossypol to boronic acid ligands associated with the NPs, and to the cooperative dissociation of the boronate ester group by their substitution with the lactate ligand. The results indicate that the stimuli-responsive gossypol boronate ester capped pores might be versatile capping units for other composite anti-cancer drug load MP-SiO₂ NPs that reveal dual chemotherapeutic functions. The procedure presented in this invention was upscaled to prepare an eight-fold quantity of the loaded NPs. These results suggest that the concept may be further upscaled to even larger scales.

### BRIEF DESCRIPTION OF THE DRAWINGS

In order to better understand the subject matter that is disclosed herein and to exemplify how it may be carried out in practice, embodiments will now be described, by way of non-limiting example only, with reference to the accompanying drawings, in which:

FIG. 1 is a schematic preparation of Methylene Blue- or Mitoxantrone-loaded Gossypol-capped boronic acid-functionalized mesoporous SiO₂ nanoparticles, MP-SiO₂ NPs, and the unlocking of the pores and the release of the loads under acidic conditions and in the presence of lactic acid.

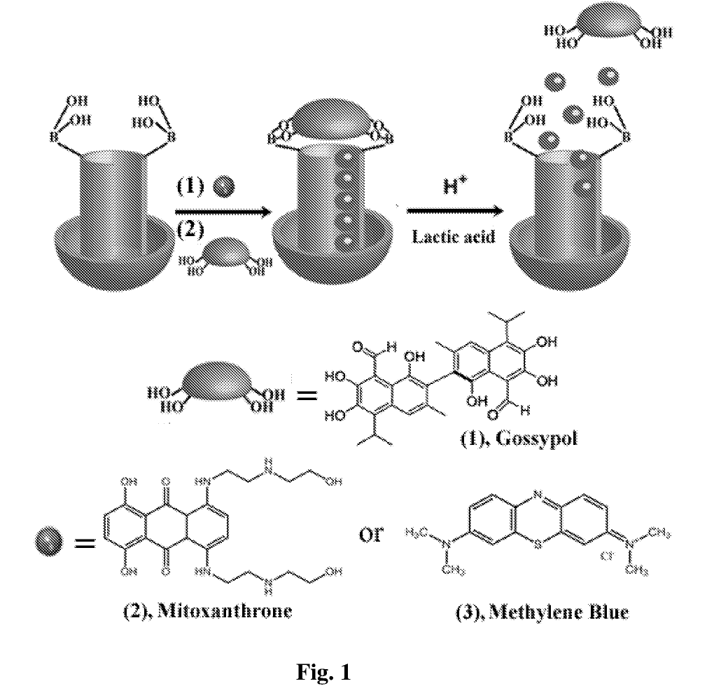

FIG. 2 shows the absorption spectra corresponding to: (a) Alizarin Red S; (b) Aminopropyl siloxane-functionalized MP-SiO₂ NPs in the presence of Alizarin Red S; (c) Boronic acid-modified MP-SiO₂ NPs in the presence of Alizarin Red S. All data were recorded in a PBS buffer solution, 200 mM, pH=7.4 in the presence of Alizarin Red S 100 μM.

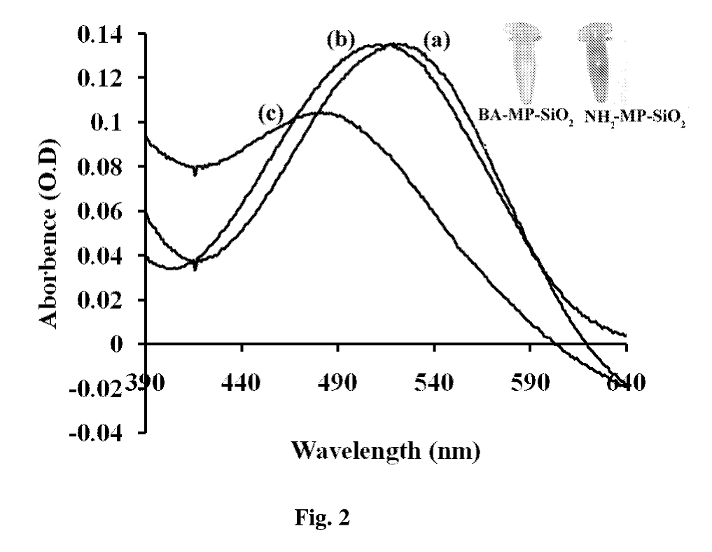

FIGS. 3A-3B shows (3A) Time-dependent fluorescence changes upon unlocking of the Methylene Blue-loaded gossypol-capped MP-SiO₂ NPs upon: (a) In the presence of the PBS buffer solution 200 mM, pH=7.4; (b) In the presence of lactic acid, 200 mM, pH=6.0; (c) In the presence of formic acid, 200 mM, pH=4.5; (d) In the presence of lactic acid, 200 mM, pH=4.5 (error bars derived from N=4 experiments). (3B) Time-dependent fluorescence changes upon unlocking of the MX-loaded gossypol-capped MP-SiO₂ NPs 200 mM, pH=7.4; (b) In the presence of lactic acid, 200 mM, pH=6.0; (c) In the presence of formic acid, 200 mM, pH=4.5; (d) In the presence of lactic acid, 200 mM, pH=4.5 (Error bars derived from N=4 experiments).

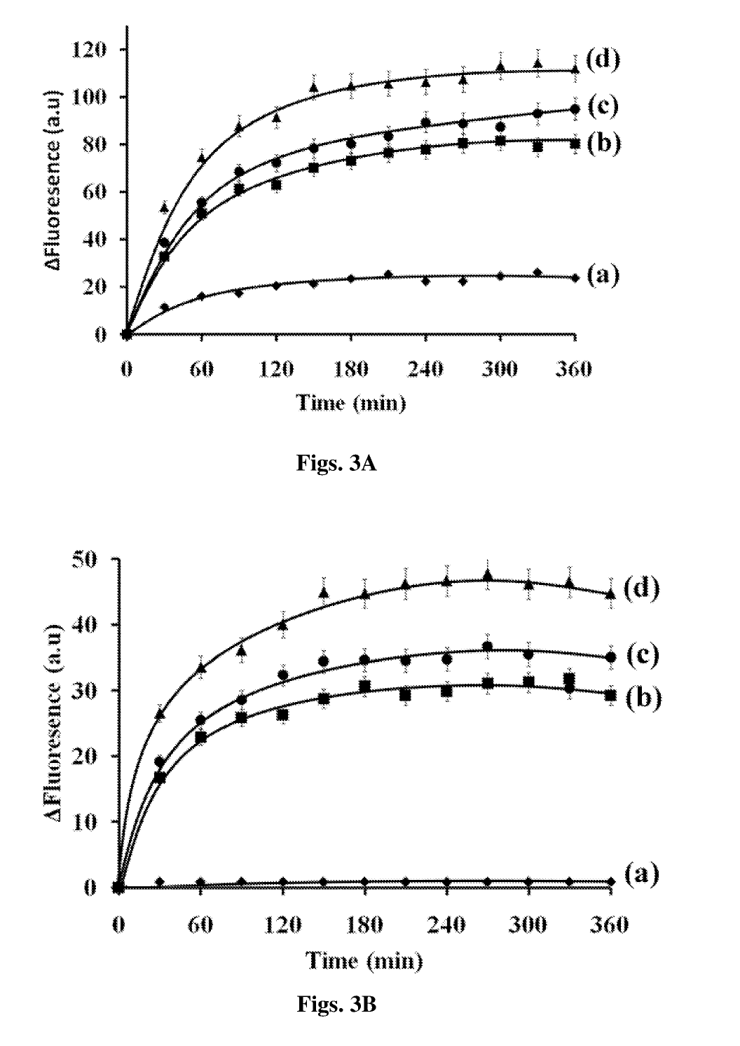

FIGS. 4A-4B shows (4A) Schematic preparation of MX-loaded β-cyclodextrin-capped boronic acid-functionalized mesoporous SiO₂ nanoparticles, MP-SiO₂ NPs, and the unlocking of the pores and the release of the loads under acidic conditions in the presence of lactic acid. (B) Time-dependent fluorescence changes upon unlocking of the Mitoxantrone-loaded β-cyclodextrin-capped MP SiO₂ NPs upon: (a) In the presence of the PBS buffer solution, 200 mM, pH=7.4; (b) In the presence of lactic acid, 200 mM, pH=6.0; (c) In the presence of formic acid, 200 mM, pH=4.5; (d) In the presence of lactic acid, 200 mM, pH=4.5 (Error bars derived from N=4 experiments).

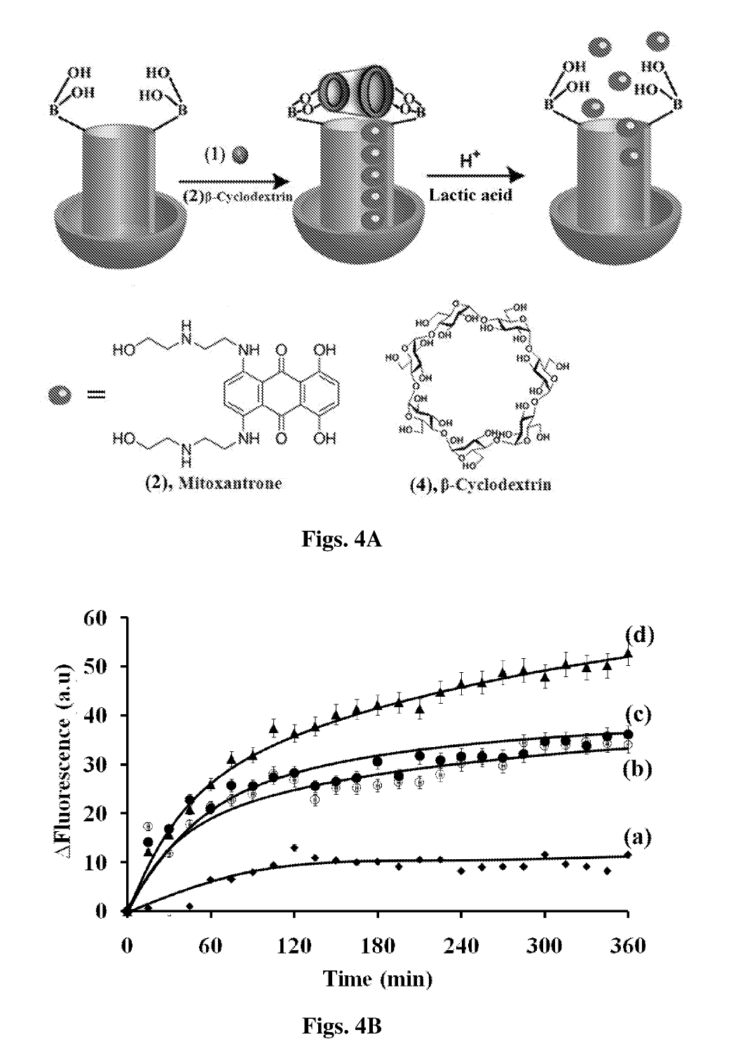

### DETAILED DESCRIPTION OF EMBODIMENTS

### Experimental Section

Materials:

Ultrapure water from NANOpure Diamond (Barnstead Int., Dubuque, Iowa) source was used throughout the experiments. Tetraethyl orthosilicate (TEOS), (3-aminopropyl) triethoxysilane (APTES) were purchased from Aldrich. All other chemicals were obtained from Sigma and were used as supplied.

Instrumentation:

Fluorescence measurements were performed using a Cary Eclipse device (Varian Inc.). UV-vis absorption spectra were recorded using a Shimadzu UV-2401 spectrophotometer. Surface areas were determined using a Nova 1200e BET meter (Quantachrome Instruments, USA) by nitrogen adsorption/desorption at the temperature of liquid nitrogen. SEM images were taken by a Sirion high resolution scanning electron microscope.

Synthesis of Mesoporous Silica Nanoparticles:

Amino-functionalized mesoporous SiO₂ NPs were prepared according to a previously reported procedure with some modifications.²⁸ The resulting NPs were precipitated, washed with distilled water and methanol, and were and dried under high vacuum (overnight). In order to remove the N-cetyltrimethylammonium bromide (CTAB), the MP-SiO₂ NPs were refluxed for 16 h in a solution composed of HCl (37%, 1 mL) and methanol (80 mL), and were, then, extensively washed with distilled water and methanol. The surfactant-free mesoporous SiO₂ material was placed under high vacuum (overnight) with heating at 60° C. to remove the remaining solvent from the mesopores. The resulting NPs (0.5 g) was refluxed for 20 h (145° C., 320 rpm) in 40.0 mL of anhydrous toluene with 0.67 mL of 3-aminopropyltrimethoxysilane (APTMS) to yield the 3-aminopropyl-functionalized mesoporous SiO₂ material. The resulting material was filtered and extensively washed with toluene, methanol, nanopure water and the purified amine-modified mesoporous SiO₂ material (400 mg) was dispersed in 20 mL dimethyl sulfoxide (DMSO). 0.15 g (0.90 mmol) 4-carboxyphenylboronic acid (CBA) was reacted with 0.10 g (0.87 mmol) N-hydroxysuccinimide (NHS) and 0.20 g (1.04 mmol) 1-ethyl-3-(3-dimethylaminopropyl) carbodiimide hydrochloride (EDC) in 5.0 mL DMSO, stirring at room temperature for 15 min before adding to the amine-modified mesoporous SiO₂ suspension. The mixture was stirred at room temperature for another 24 h, followed by filtration and washing with DMSO, water and methanol. The BA-MSN material was placed under high vacuum (over night). The coverage of the amine-functionalities on the MP-SiO₂ NPs was evaluated by the ninhydrin test²⁹ to be 5.7 nmole·gr⁻¹, and surface boronic acid groups were calculated to be around 0.5 mmol/g by subtracting the amount of remaining surface amine groups from that on amine-modified mesoporous SiO₂ surface. The AP-MSN material was placed under high vacuum (overnight).

Loading and Release of the Drugs:

The mesoporous SiO₂ NPs (10 mg) were dispersed in 1 ml PBS saline and sonicated for 20 min. The MP-SiO₂ NPs were loaded with 100 μl (10 mM) anti-cancer drug mitoxantrone. The solution was gently shaken for overnight.

The loaded NPs were capped with 30 μl gossypol (30 mg/ml in DMF) or 60 μl β-cyclodextrin (0.05M in CHES buffer, pH=9.8) and the solution was gently shaken for overnight.

The loaded NPs capped with gossypol and the mitoxantrone substrate associated with surface domain or uncapped pores were washed off with methanol ×50 and with TDW ×25 and then were lyophilized.

The unlocking of the capped MP-SiO₂ NPs and the release of mitoxantrone was examined under conditions that could simulate the unlocking process in native cancer cell environments, in the presence of lactic acid, 200 mM, at pH=6.0 and pH=4.5 at 37° C. after a time-interval of 24 hours.

1.-22. (canceled)

23. Mesoporous nanoparticle (MP-NP) loaded within its pores with at least one loaded pharmaceutically active agent; comprising at least one ligand baring boronic acid or derivative thereof on the surface of said pores chemically coordinated with at least one capping pharmaceutically active agent.

24. The MP-NP of claim 23, being selected from silica, alumina, zirconia, titania, carbon nanoparticle, and any combinations thereof.

25. The MP-NP of claim 23, wherein said at least one loaded pharmaceutically active agent is selected an anti-cancer agent.

26. The MP-NP of claim 23, wherein said at least one loaded pharmaceutically active agent is selected mitoxantrone.

27. The MP-NP of claim 23, wherein said at least one capping pharmaceutically active agent is an anti-cancer agent.

28. The MP-NP of claim 23, wherein said at least one capping pharmaceutically active agent is gossypol.

29. The MP-NP of claim 23, wherein said at least one capping pharmaceutically active agent is cyclodextrin.

30. The MP-NP of claim 23, further comprising at least one ligand on the surface of said nanoparticle chemically coordinated with at least one further active agent.

31. The MP-NP of claim 23, capable of triggered simultaneous release of said at least one loaded pharmaceutically active agent and said at least one capping pharmaceutically active agent.

32. The MP-NP of claim 23, capable of triggered simultaneous controlled release of said at least one loaded pharmaceutically active agent and said at least one capping pharmaceutically active agent.

33. A composition comprising at least one mesoporous nanoparticle (MP-NP) loaded within its pores with at least one loaded pharmaceutically active agent; comprising at least one ligand bring boronic acid or derivative thereof on the surface of said pores chemically coordinated with at least one capping pharmaceutically active agent.

34. A method of treating cancer in a patient suffering therefrom, comprising administering to said patient a composition comprising at least one mesoporous nanoparticle (MP-NP) loaded within its pores with at least one loaded pharmaceutically active agent; comprising at least one ligand bring boronic acid or derivative thereof on the surface of said pores chemically coordinated with at least one capping pharmaceutically active agent.
